(19) United States
(12) Patent Application Publication
(10) Pub. No.: US 2022/0310258 A1
(43) Pub. Date: Sep. 29, 2022

(54) PERSONALIZED LOCATION RECOMMENDATION FOR MEDICAL PROCEDURES

(71) Applicant: International Business Machines Corporation, Armonk, NY (US)

(72) Inventors: Stan Kevin Daley, Atlanta, GA (US); Michael Bender, Rye Brook, NY (US)

(21) Appl. No.: 17/209,803

(22) Filed: Mar. 23, 2021

(51) Int. Cl. G16H 50/20; G16H 70/20; G16H 10/60; G16H 50/70; G16H 15/00; G16H 40/67; G06F 3/14

(52) U.S. Cl. CPC G16H 50/20; G16H 70/20; G16H 10/60; G16H 50/70; G16H 15/00; G16H 40/67; G06F 3/14; G16H 20/17

(57) ABSTRACT

A processor may receive procedure data regarding a medical procedure. The processor may receive patient data regarding a patient intended to receive the medical procedure. The processor may identify, using an artificial intelligence (AI) model, features of the data related to performance of the medical procedure on the patient. The processor may analyze the relation of the features to locations for performance of the medical procedure. The processor may determine one or more locations for the performance of the medical procedure. The processor may output the one or more locations for the performance of the medical procedure to a display.

### BACKGROUND

The present disclosure relates generally to the field of providing location recommendations for medical procedures, and more specifically to providing personalized location recommendations for medical procedures based on cognitive analysis.

Venipuncture is a skill taught to phlebotomists and other medical practitioners for drawing blood from an individual or infusing medication through needles inserted into a vein. Some individuals receive regular or frequent venipuncture procedures.

### SUMMARY

Embodiments of the present disclosure include a method, computer program product, and system for providing location recommendations for medical procedures. A processor may receive procedure data regarding a medical procedure. The processor may receive patient data regarding a patient intended to receive the medical procedure. The processor may identify, using an artificial intelligence (AI) model, features of the data related to performance of the medical procedure on the patient. The processor may analyze the relation of the features to locations for performance of the medical procedure. The processor may determine one or more locations for the performance of the medical procedure. The processor may output the one or more locations for the performance of the medical procedure to a display.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a block diagram of an exemplary system for providing location recommendations for medical procedures, in accordance with aspects of the present disclosure.

FIG. 2 is a flowchart of an exemplary method system for providing location recommendations for medical procedures, in accordance with aspects of the present disclosure.

FIG. 3A illustrates a cloud computing environment, in accordance with aspects of the present disclosure.

FIG. 3B illustrates abstraction model layers, in accordance with aspects of the present disclosure.

FIG. 4 illustrates a high-level block diagram of an example computer system that may be used in implementing one or more of the methods, tools, and modules, and any related functions, described herein, in accordance with aspects of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of providing location recommendations for medical procedures, and more specifically to providing personalized location recommendations for medical procedures based on cognitive analysis. While the present disclosure is not necessarily limited to such applications, various aspects of the disclosure may be appreciated through a discussion of various examples using this context.

Venipuncture is a skill taught to phlebotomists and other medical practitioners for drawing blood from an individual or infusing medication through needles inserted into a vein. Some individuals receive regular or frequent venipuncture procedures. The individuals that frequently need these treatments can have negative responses if a location is chosen too frequently or their veins are collapsing.

In some embodiments, a processor may receive procedure data regarding a medical procedure. In some embodiments, the procedure data may relate to the identity and/or purpose of a planned medical procedure. In some embodiments, the medical procedures may relate to venipuncture. In some embodiments, the venipuncture procedures may be for the purpose of obtaining blood for diagnostic purposes, monitoring levels of blood components, administering therapeutic treatments (e.g., medication, nutrition, or chemotherapy), removing blood due to excess levels of iron or erythrocytes, or collecting blood for later uses (e.g., transfusions), etc. For example, the processor may receive data regarding a venipuncture procedure to obtain blood for diagnostic purposes. In some embodiments, the procedure data may include description of characteristics or specific aspects of intended procedure. For example, the data may include a description that the blood is being drawn for a complete blood count test that is used to evaluate an individual's overall health and detect a wide range of aliments (as opposed to blood being drawn for a donation purposes).

In some embodiments, the processor may receive patient data regarding a patient intended to receive the medical procedure. In some embodiments, the patient data may include information that is relevant to or related to the performance of the medical procedure, the medical condition of the patient, the physical condition of the patient, etc. In some embodiments, the patient data may include age, health condition, height, medical history, biometrics (percent water in blood, hematocrit level, etc.), etc. For example, the patient data may indicate that the patient has high iron count and receives regular blood withdrawals every month. The patient data may indicate that the patient is an elderly cancer patient who receives frequent venipuncture for hydration, medication, and blood tests. In some embodiments, the patient data is received via opt-in consent from the patient.

In some embodiments, the patient data may include the history of the patient with previous procedures that are the same as or similar to the intended medical procedure. For example, the patient may have previously received venipuncture procedures for the receiving of IV fluids and is now planned to receive venipuncture for receiving intravenous medication. The patient data may include information regarding the patient's experience receiving IV fluids, including time of last procedure, location of last procedure, basis for selection of location (e.g., patient preferred selection of non-dominant arm, other arm was in a cast and not accessible, other locations were tested first but failed, etc.), details about how the previous procedure was implemented (e.g., with a needle of a certain size, without asking the patient to drink additional water, after using a tourniquet to improve the visibility of the veins), the outcome or quality of the procedure (e.g., adverse events, verbalization by patient that the procedure caused soreness, bruising, successful withdraw blood a pint of blood, blood withdrawal was unsuccessful after flow volume decreased, etc.), etc.

In some embodiments, the processor may identify, using an artificial intelligence (AI) model, features of the data related to performance of the medical procedure on the patient. In some embodiments, the processor may analyze the relation of the features to locations for performance of the medical procedure. In some embodiments, the AI model may be trained using information from medical documents (e.g., textbooks, publications, medical images) regarding the recommended locations for venipuncture, recommended procedures for obtaining the best outcome for patients (e.g., follow up steps), and practices or locations to avoid. In some embodiments, the AI model may be trained using data regarding historical insertion locations (e.g., prior patients' medical histories), reactions or outcomes from the use of the insertion location, details of the techniques used, and background context associated with the selection of the location and/or the outcome or reaction to use of the location. For example, the AI model may be trained with information from medical textbooks that indicate that a patient's dominant arm should be used for venipuncture involving blood extraction, and a patient's non-dominant arm should be used for procedures introducing medications into the patient. In some embodiments, the AI model may be trained to make predictions regarding the frequency, timing, and type of additional venipuncture procedures the patient may receive in the future (e.g., if the patient regularly receives IV nutrients or medications) to recommend locations for performance of the procedure on this occasion.

In some embodiments, the features may relate to performance of the medical procedure on the patient. In some embodiments, the features may relate to performance of the medical procedure on the patient personalized to the physical and medical characteristics of the patient. In some embodiments, the features may relate to performance of the medical procedure on the patient personalized to past experiences of the patient with the same or similar medical procedures. In some embodiments, the features may relate to preferred location and techniques for the medical procedure that take into account past experiences of the patient with the same or similar medical procedures. In some embodiments, the features may relate to preferred location and technique that take into account the patients prior venipuncture experiences, including the prior location, prior technique, prior reaction to technique, prior outcome from technique, time of last technique, basis for selection of prior location, etc.

In some embodiments, the processor may determine one or more locations for the performance of the medical procedure. In some embodiments, the processor may output the one or more locations for the performance of the medical procedure to a display. In some embodiments, the one or more locations may include locations on the patient's body that are preferred for the performance of the medical procedure based on the historical data used to train the AI model and the patient's history with the same or similar medical procedures. In some embodiments, the one or more locations may be a point or an area of the body preferred for the medical procedure. In some embodiments, the one or more preferred locations displayed may include an image of a body part tagged/marked with one or more locations. In some embodiments, the one or more preferred locations displayed may include a textual description of the location (e.g., five cm below the elbow). In some embodiments, the display may include an interface of a computing device, a video screen, a computer screen, a display of a tablet device, a projector display, etc.

In some embodiments, the display may be an augmented reality display. For example, the augmented reality display may include augmented reality glasses in communication with a computing device. A medical professional may see the patient's body part through the augmented reality glasses. The medical professional may see the preferred locations overlayed on the regions of the patient's body that are recommended (e.g., the preferred locations may be colored green) as the medical professional aims for that part of the body to perform the venipuncture. In some embodiments, the augmented reality display may include filters or light settings to help the medical professional more easily see the patient's veins.

In some embodiments, the one or more locations may include a first preferred location and a second preferred location. In some embodiments, the processor may determine a priority of the first preferred location with respect to the second preferred location. In some embodiments, the processor may display the priority of the first preferred location with respect to the second preferred location. In some embodiments, the first preferred location may be given higher priority (e.g., recommended to be tried first) over the second preferred location based on historical data, the history of the patient with the same or similar medical procedures, or mental/physical attributes of the patient. In some embodiments, the priority of the first preferred location with respect to the second preferred location may be output by an AI model or an AI technology such as IBM Watson Health. In some embodiments, the priority may be displayed by textual indicators, tags, different symbols indicating a first choice and second choice, color coding, shading of the regions of the body, etc. In some embodiments, multiple locations may be output by the processor. As an example, the priority for using the multiple locations may be displayed by shading different areas on an image of the arm associated with veins of the patient with different shades of green to indicate a priority of the different areas for venipuncture.

In some embodiments, the processor may determine one or more locations that are to be avoided for the performance of the medical procedure. In some embodiments, the processor may display the one or more locations that are to be avoided for the performance of the medical procedure. For example, the processor may determine that a location that was used for a recent prior medical procedure should be avoided. The location to be avoided may be shown on the display using text, tags indicating that this is a location to be avoided, symbols indicating that this is a location to be avoided, color coding, etc.

In some embodiments, the processor may provide instructions regarding preferred techniques for performance of the medical procedure to a user. In some embodiments, the preferred techniques may relate to variations in ways to perform the medical procedure (e.g., insert the needle with a 45 degree angle rather than a 30 degree angle) or additional steps to perform the procedure (e.g., instruct the patient to drink an additional glass of water one hour before the procedure or wait until the patient's blood pressure has decreased). In some embodiments, the preferred techniques may be provided to the medical professional (e.g., on the augmented reality display) or may be provided to the patient (e.g., using an audio output).

In some embodiments, the processor may receive data from a recording device regarding the performance of the medical procedure on the patient. In some embodiments, the processor may provide the data to the AI model. In some embodiments, the recording device may be a video camera, GoPro, tablet camera, etc. In some embodiments, the recording device may observe the locations used during the venipuncture procedure. In some embodiments, the recording device may observe outcomes from the medical procedure (e.g., the patient's face indicated the patient was in pain). In some embodiments, the recording device may obtain data regarding how the procedure was performed (e.g., the medical professional attempted five times to hit the vein with a needle before finding the correct location or the patient had to pump her fists multiple time to achieve blood flow). In some embodiments, the recording device may observe physical attributes of the patient (e.g., the patient's veins were thin, the patient was making a fist, etc.). In some embodiments, a patient or medical professional may input feedback about the performance of the medical procedure (e.g., it caused pain) into a graphical user interface on a computing device.

In some embodiments, the processor may provide the data to the AI model for the AI model to provide improved recommendations for locations for performance of future medical procedures. The AI model may use the data received from the recording device to identify features of the data related to performance of the medical procedure on the patient, analyze the relation of the features to locations for performance of the medical, and determine one or more locations for the performance of the medical procedure.

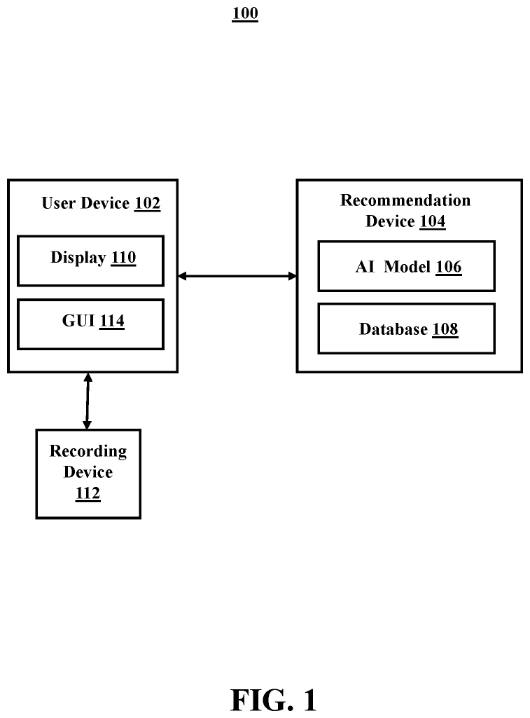

Referring now to FIG. 1, a block diagram of a system 100 for providing location recommendations for medical procedures is illustrated. System 100 includes a user device 102 and a recommendation device 104 (e.g., server). The recommendation device 104 is configured to be in communication with the user device 102. The recommendation device 104 includes an AI model 106 and a database 108. The database 108 stores the procedure data, patient data, and the data used to train the AI model. In some embodiments, the user device 102 and the recommendation device 104 may be any devices that contain a processor configured to perform one or more of the functions or steps described in this disclosure.

In some embodiments, a user (e.g., medical professional) enters procedure data regarding the medical procedure intended to be performed and patient data regarding the patient intended to receive the medical procedure into the user device 102. The procedure data and patient data is communicated from the user device 102 to the recommendation device 104. The AI model 106 of the recommendation device 104 receives the procedure data and the patient data and identifies features of the data related to performance of the medical procedure on the patient. The AI model 106 analyzes the relation of the features to locations for performance of the medical procedure and determines one or more locations for the performance of the medical procedure. The recommendation device 104 communicates the one or more locations to the user device 102, and the one or more locations for performing the medical procedure are displayed on the display 110 of the user device 102. In some embodiments, the recommendation device 104 may provide instructions regarding preferred techniques for performing the medical procedure to the user device 102 and those instructions may be shown on the display 110.

In some embodiments, the display 110 may be an augmented reality display allowing the user to see the patient's body part overlayed with a marker indicating the preferred locations for performing the medical procedure.

In some embodiments, the recommendation device 104 may determine a priority of the first preferred location with respect to the second preferred location. In some embodiments, the priority of the first preferred location with respect to the second preferred location may be displayed on display 110 of the user device 102. In some embodiments, the recommendation device 104 may determine one or more locations that are to be avoided for the performance of the medical procedure. In some embodiments, the one or more locations that are to be avoided for the performance of the medical procedure may be displayed on display 110 of the user device 102.

In some embodiments, the recommendation device 104 may receiving data from a recording device 112 in communication with user device 102 regarding the performance of the medical procedure on the patient. In some embodiments, the recommendation device 104 may provide the data to the AI model to further optimize the personalize recommendation for the patient for the location for performance of future medical procedures. In some embodiments, a patient or medical professional may input feedback about the performance of the medical procedure (e.g., it caused pain) into a graphical user interface 114 on the user device 102.

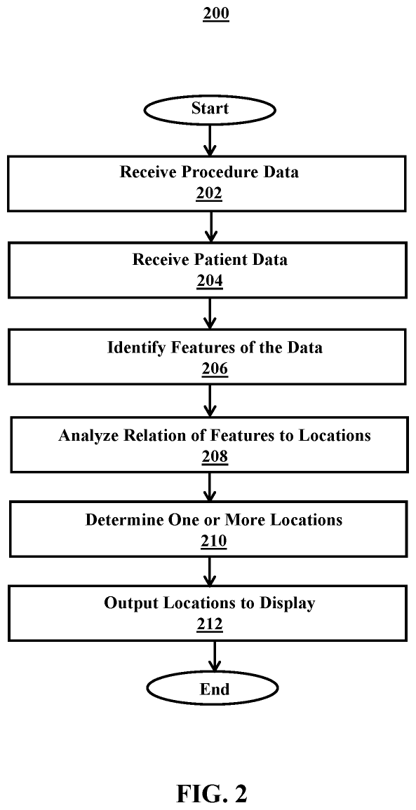

Referring now to FIG. 2, illustrated is a flowchart of an exemplary method 200 for providing location recommendations for medical procedures, in accordance with embodiments of the present disclosure. In some embodiments, a processor of a system may perform the operations of the method 200. In some embodiments, method 200 begins at operation 202. At operation 202, the processor receives procedure data regarding a medical procedure. In some embodiments, method 200 proceeds to operation 204, where the processor receives patient data regarding a patient intended to receive the medical procedure. In some embodiments, method 200 proceeds to operation 206. At operation 206, the processor identifies, using an AI model, features of the data related to performance of the medical procedure on the patient. In some embodiments, method 200 proceeds to operation 208. At operation 208, the processor analyzes the relation of the features to locations for performance of the medical procedure. In some embodiments, method 200 proceeds to operation 210. At operation 210, the processor determines one or more locations for the performance of the medical procedure. In some embodiments, method 200 proceeds to operation 212. At operation 212, the processor outputs the one or more locations for the performance of the medical procedure to a display.

As discussed in more detail herein, it is contemplated that some or all of the operations of the method 200 may be performed in alternative orders or may not be performed at all; furthermore, multiple operations may occur at the same time or as an internal part of a larger process.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

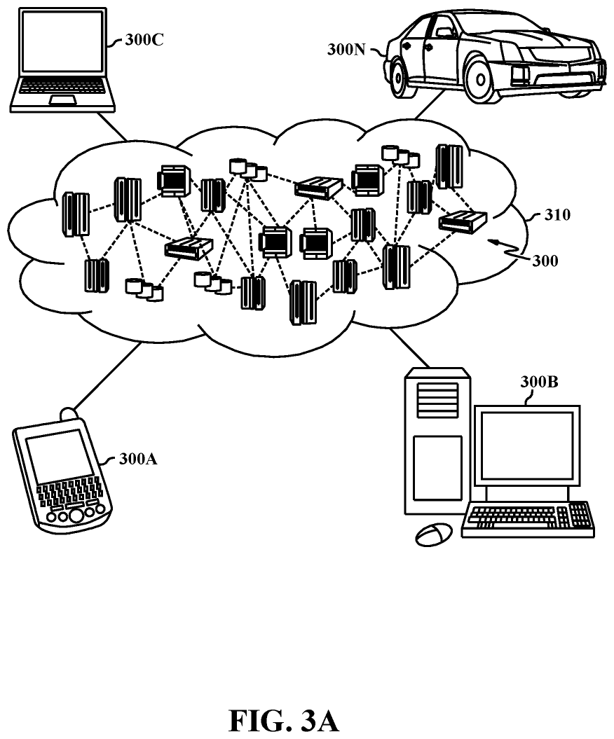

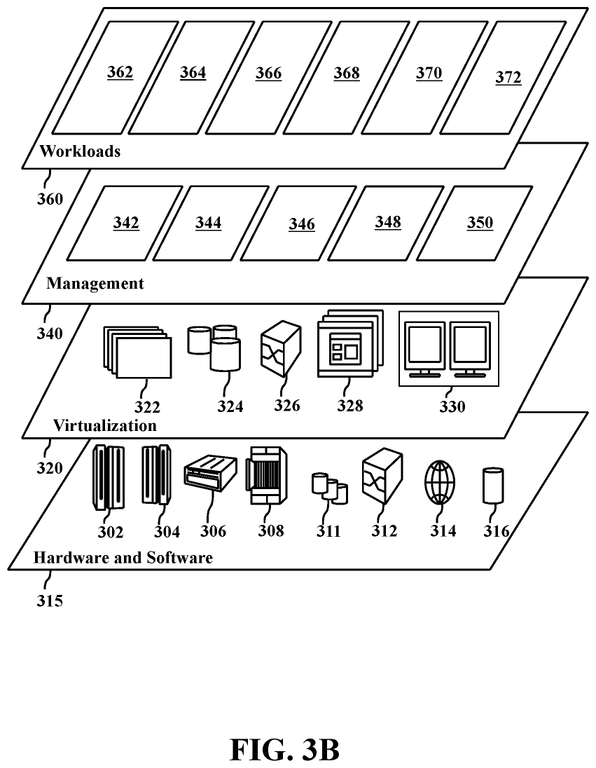

FIG. 3A, illustrated is a cloud computing environment 310 is depicted. As shown, cloud computing environment 310 includes one or more cloud computing nodes 300 with which local computing devices used by cloud consumers, such as, for example, personal digital assistant (PDA) or cellular telephone 300A, desktop computer 300B, laptop computer 300C, and/or automobile computer system 300N may communicate. Nodes 300 may communicate with one another. They may be grouped (not shown) physically or virtually, in one or more networks, such as Private, Community, Public, or Hybrid clouds as described hereinabove, or a combination thereof.

This allows cloud computing environment 310 to offer infrastructure, platforms and/or software as services for which a cloud consumer does not need to maintain resources on a local computing device. It is understood that the types of computing devices 300A-N shown in FIG. 3A are intended to be illustrative only and that computing nodes 300 and cloud computing environment 310 can communicate with any type of computerized device over any type of network and/or network addressable connection (e.g., using a web browser).

FIG. 3B, illustrated is a set of functional abstraction layers provided by cloud computing environment 310 (FIG. 3A) is shown. It should be understood in advance that the components, layers, and functions shown in FIG. 3B are intended to be illustrative only and embodiments of the disclosure are not limited thereto. As depicted below, the following layers and corresponding functions are provided.

Hardware and software layer 315 includes hardware and software components. Examples of hardware components include: mainframes 302; RISC (Reduced Instruction Set Computer) architecture based servers 304; servers 306; blade servers 308; storage devices 311; and networks and networking components 312. In some embodiments, software components include network application server software 314 and database software 316.

Virtualization layer 320 provides an abstraction layer from which the following examples of virtual entities may be provided: virtual servers 322; virtual storage 324; virtual networks 326, including virtual private networks; virtual applications and operating systems 328; and virtual clients 330.

In one example, management layer 340 may provide the functions described below. Resource provisioning 342 provides dynamic procurement of computing resources and other resources that are utilized to perform tasks within the cloud computing environment. Metering and Pricing 344 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 346 provides access to the cloud computing environment for consumers and system administrators. Service level management 348 provides cloud computing resource allocation and management such that required service levels are met. Service Level Agreement (SLA) planning and fulfillment 350 provide pre-arrangement for, and procurement of, cloud computing resources for which a future requirement is anticipated in accordance with an SLA.

Workloads layer 360 provides examples of functionality for which the cloud computing environment may be utilized. Examples of workloads and functions which may be provided from this layer include: mapping and navigation 362; software development and lifecycle management 364; virtual classroom education delivery 366; data analytics processing 368; transaction processing 370; and providing location recommendations for medical procedures 372.

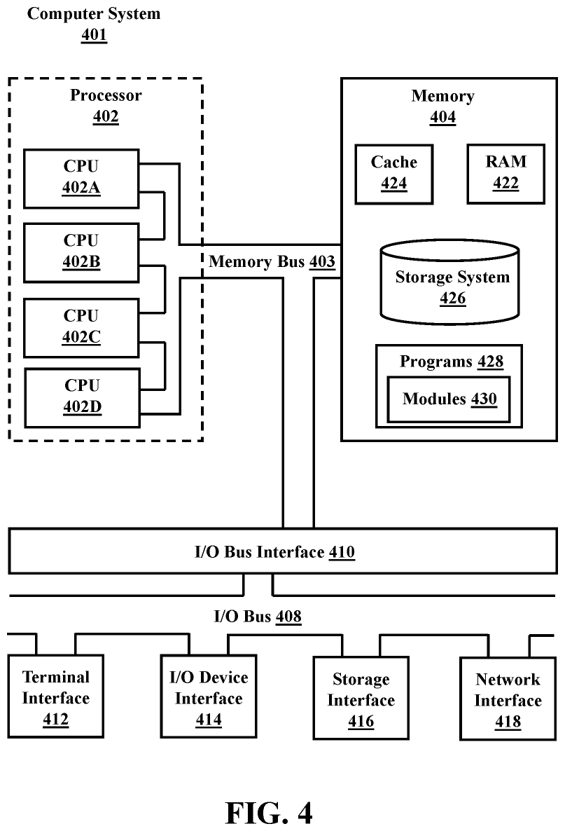

FIG. 4, illustrated is a high-level block diagram of an example computer system 401 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 401 may comprise one or more CPUs 402, a memory subsystem 404, a terminal interface 412, a storage interface 416, an I/O (Input/Output) device interface 414, and a network interface 418, all of which may be communicatively coupled, directly or indirectly, for inter-component communication via a memory bus 403, an I/O bus 408, and an I/O bus interface unit 410.

The computer system 401 may contain one or more general-purpose programmable central processing units (CPUs) 402A, 402B, 402C, and 402D, herein generically referred to as the CPU 402. In some embodiments, the computer system 401 may contain multiple processors typical of a relatively large system; however, in other embodiments the computer system 401 may alternatively be a single CPU system. Each CPU 402 may execute instructions stored in the memory subsystem 404 and may include one or more levels of on-board cache.

System memory 404 may include computer system readable media in the form of volatile memory, such as random access memory (RAM) 422 or cache memory 424. Computer system 401 may further include other removable/non-removable, volatile/non-volatile computer system storage media. By way of example only, storage system 426 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 404 can include flash memory, e.g., a flash memory stick drive or a flash drive. Memory devices can be connected to memory bus 403 by one or more data media interfaces. The memory 404 may include at least one program product having a set (e.g., at least one) of program modules that are configured to carry out the functions of various embodiments.

One or more programs/utilities 428, each having at least one set of program modules 430 may be stored in memory 404. The programs/utilities 428 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 428 and/or program modules 430 generally perform the functions or methodologies of various embodiments.

Although the memory bus 403 is shown in FIG. 4 as a single bus structure providing a direct communication path among the CPUs 402, the memory subsystem 404, and the I/O bus interface 410, the memory bus 403 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 410 and the I/O bus 408 are shown as single respective units, the computer system 401 may, in some embodiments, contain multiple I/O bus interface units 410, multiple I/O buses 408, or both. Further, while multiple I/O interface units are shown, which separate the I/O bus 408 from various communications paths running to the various I/O devices, in other embodiments some or all of the I/O devices may be connected directly to one or more system I/O buses.

In some embodiments, the computer system 401 may be a multi-user mainframe computer system, a single-user system, or a server computer or similar device that has little or no direct user interface, but receives requests from other computer systems (clients). Further, in some embodiments, the computer system 401 may be implemented as a desktop computer, portable computer, laptop or notebook computer, tablet computer, pocket computer, telephone, smartphone, network switches or routers, or any other appropriate type of electronic device.

It is noted that FIG. 4 is intended to depict the representative major components of an exemplary computer system 401. In some embodiments, however, individual components may have greater or lesser complexity than as represented in FIG. 4, components other than or in addition to those shown in FIG. 4 may be present, and the number, type, and configuration of such components may vary.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

What is claimed is:

1. A computer-implemented method, the method comprising:
receiving, by a processor, procedure data regarding a medical procedure;
receiving patient data regarding a patient intended to receive the medical procedure;
identifying, using an artificial intelligence (AI) model, features of the data related to performance of the medical procedure on the patient;
analyzing the relation of the features to locations for performance of the medical procedure;
determining one or more locations for the performance of the medical procedure; and
outputting the one or more locations for the performance of the medical procedure to a display.

2. The method of claim 1, wherein the display includes an augmented reality display.

3. The method of claim 1, wherein the one or more locations include a first preferred location and a second preferred location, and wherein the method further comprises:
determining a priority of the first preferred location with respect to the second preferred location; and
displaying the priority of the first preferred location with respect to the second preferred location.

4. The method of claim 1, wherein determining one or more locations for the performance of the medical procedure includes:
determining one or more locations that are to be avoided for the performance of the medical procedure; and
displaying the one or more locations that are to be avoided for the performance of the medical procedure.

5. The method of claim 1, further comprising:
providing instructions regarding preferred techniques for performance of the medical procedure to a user.

6. The method of claim 1, further comprising:
receiving data from a recording device regarding the performance of the medical procedure on the patient; and
providing the data to the AI model.

7. A system comprising:
a memory; and
a processor in communication with the memory, the processor being configured to perform operations comprising:
receiving procedure data regarding a medical procedure;
receiving patient data regarding a patient intended to receive the medical procedure;
identifying, using an artificial intelligence (AI) model, features of the data related to performance of the medical procedure on the patient;
analyzing the relation of the features to locations for performance of the medical procedure;
determining one or more locations for the performance of the medical procedure; and
outputting the one or more locations for the performance of the medical procedure to a display.

8. The system of claim 7, wherein the display includes an augmented reality display.

9. The system of claim 7, wherein the one or more locations include a first preferred location and a second preferred location, and wherein the processor is further configured to perform operations comprising:
determining a priority of the first preferred location with respect to the second preferred location; and
displaying the priority of the first preferred location with respect to the second preferred location.

10. The system of claim 7, wherein determining one or more locations for the performance of the medical procedure includes:
determining one or more locations that are to be avoided for the performance of the medical procedure; and
displaying the one or more locations that are to be avoided for the performance of the medical procedure.

11. The system of claim 7, the processor being further configured to perform operations comprising:
providing instructions regarding preferred techniques for performance of the medical procedure to a user.

12. The system of claim 7, the processor being further configured to perform operations comprising:
receiving data from a recording device regarding the performance of the medical procedure on the patient; and
providing the data to the AI model.

13. A computer program product comprising a computer readable storage medium having program instructions embodied therewith, the program instructions executable by a processor to cause the processor to perform operations, the operations comprising:
receiving procedure data regarding a medical procedure;
receiving patient data regarding a patient intended to receive the medical procedure;
identifying, using an artificial intelligence (AI) model, features of the data related to performance of the medical procedure on the patient;
analyzing the relation of the features to locations for performance of the medical procedure;
determining one or more locations for the performance of the medical procedure; and
outputting the one or more locations for the performance of the medical procedure to a display.

14. The computer program product of claim 13, wherein the display includes an augmented reality display.

15. The computer program product of claim 13, wherein the one or more locations include a first preferred location and a second preferred location, and wherein the processor is further configured to perform operations comprising:
determining a priority of the first preferred location with respect to the second preferred location; and
displaying the priority of the first preferred location with respect to the second preferred location.

16. The computer program product of claim 13, wherein determining one or more locations for the performance of the medical procedure includes:
determining one or more locations that are to be avoided for the performance of the medical procedure; and
displaying the one or more locations that are to be avoided for the performance of the medical procedure.

17. The computer program product of claim 13, the processor being further configured to perform operations comprising:
providing instructions regarding preferred techniques for performance of the medical procedure to a user.

18. The computer program product of claim 13, the processor being further configured to perform operations comprising:
receiving data from a recording device regarding the performance of the medical procedure on the patient; and
providing the data to the AI model.
